(19) United States
(12) Patent Application Publication
(10) Pub. No.: US 2012/0088909 A1
(43) Pub. Date: Apr. 12, 2012

(54) Method for producing polysaccharide derivatives

(75) Inventors: André Lehmann, Potsdam (DE); Bert Volkert, Berlin (DE)

(73) Assignee: Fraunhofer-Gesellschaft zur Forderung der angewandten Forschung E.V., Munich (DE)

(21) Appl. No.: 13/138,557

(22) PCT Filed: Mar. 2, 2010
(86) PCT No.: PCT/EP2010/052587

§ 371 Date: Dec. 7, 2011

(30) Foreign Application Priority Data
Mar. 6, 2009 (DE) 10 2009 012 161.7

(51) Int. Cl. C08B 3/00; C07H 11/00; B01J 19/12; C08B 31/02

(52) U.S. Cl. 536/58; 536/107; 536/115; 204/157.68

(57) ABSTRACT

Process for esterifying, etherifying or silylating polysaccharide or derivatives thereof by means of an esterifying reagent, etherifying reagent or silylating reagent in the presence of an ionic liquid, in which

(a) the process is carried out under heterogeneous reaction conditions, and
(b) the amount of ionic liquid is 2% to 25% by weight, based on the polysaccharide or derivatives thereof.

The present invention relates to a process for derivatizing polysaccharides or their related structures using ionic liquids.

Polysaccharides as natural polymers, and also chemically and physically modified polysaccharides, are increasingly gaining importance in a wide variety of different sectors of industry.

In chemical derivatizations of cellulose in particular, solvent systems such as, for example, N,N-dimethylacetamide-LiCl (see El Seoud, O. A. Marson, Macromolecular Chemistry and Physics, 2000, 882) or dimethyl sulphoxide/TBAF (see T. Heinze, R. Dicke, A. Koschella, Macromolecular Chemistry and Physics, 2000, 201, 627) are frequently used for a homogeneous synthesis regime. Disadvantages of such a reaction regime are various side reactions and also the work-up difficulties posed by the solvents used. Furthermore, the maximum concentration at which the polymer to be derivatized could be used is very low, being less than 20% by weight.

Ionic liquids (ILs) such as, for example, 1-N-butyl-3-methylimidazolium chloride (BmimCl) (see T. Heinze, S. Barthel, Green Chemistry, 2006, 8, 301), 1-N-allyl-3-methylimidazolium chloride (AmimCl) (see Y. Cao, J. Wu, T. Meng. Carbohydrate Polymers, 2007, 69, 665) or 1-N-ethyl-3-methylimidazolium chloride (EmimCl) have increasingly gained in importance in recent years as solvents for cellulose. Only a few papers (A. Biswas, R. L. Shogren, Carbohydrate Polymers 2006, 66 546 and D. G. Stevenson, A. Biswas, Carbohydrate Polymers, 2007, 67, 21) and also patents (WO 2007/147813 and WO 2005/023873) also consider the use of ionic liquids for the chemical reaction of further polysaccharides such as starch, for example. The use of ionic liquids as reaction medium already makes it possible to avoid aforementioned disadvantages of the existing solvents. Nevertheless, the use of ionic liquids for syntheses of polysaccharide derivatives on a larger scale is limited by factors such as an environmentally hazardous and toxic classification of a wide variety of ionic liquids and also in particular by the high price.

For the synthesis of polysaccharide esters such as cellulose acetates and starch acetates, for example, suitable pathways are shown in T. Mark, Mehltretter, Starch/Stärke, 1972, 3, 73; T. Heinze Liebert, Cellulose, 2003, 10, 283; and also in WO 98/07755. In the majority of these texts, corresponding acid chlorides or acid anhydrides are employed as esterifying reagents, with acidic or basic catalysts being employed; see, inter alia, G. Reinisch, U. Radics, Die Angewandte Makromolekulare Chemie, 1999, 4070, 113. The same is true for the industrial synthesis of cellulose acetate, as in A. Hummel, Macromolecular Symposia, 2004, 208, 61.

The preparation of polysaccharide ethers such as 2-hydroxyalkylstarches, for example, or the silylation of celluloses, typically requires that the polysaccharide be activated beforehand. In the case of starch, this can be done simply by dissolving it in an aqueous alkaline medium, after which the etherifying reagent can be added (see F. Bien, B. Wiege, Starch/Stärke, 2001, 53, 301).

The activation of cellulose, in contrast, tends to be more involved. For the silylation of cellulose, prior activation by liquid ammonia is necessary (see W. Mormann, Cellulose, 2003, 10, 271). The silylating agent is then introduced into this heterogeneous system, and the cellulose is silylated. The step of the activation of cellulose for the purpose of conversion by means of silylating agent can also be accomplished homogeneously, by dissolving the cellulose in an ionic liquid and with subsequent reaction to a silyl cellulose derivative (see WO 2007/056044). A disadvantage of this process is the limited solubility of the cellulose in the ionic liquid, resulting in the aforementioned limitations.

The use of ionic liquids as catalysts is known especially in inorganic chemistry but also in organic chemistry (see P. Wasserscheid, T. Welton, Ionic liquids in synthesis, 2nd Edition, Volume 2, 2008 and H. Zhang, F. Xu, Green Chemistry, 2007, 9, 1208). In polysaccharide chemistry, for esterification, etherification and hydrolysis reactions, publications can be found in which the native polymer is dissolved in the ionic liquid and there is therefore a high mass fraction of ionic liquid as a result of the limited solubility of the polymer in the ionic liquid.

It is an object of the present invention, therefore, to provide a process for the esterification, etherification or silylation of polysaccharides or derivatives thereof that makes it possible to achieve targeted derivatization or complete substitution of the hydroxyl groups using small amounts of liquid reaction medium. A further object of the present invention is to allow simple work-up of the substituted polysaccharides, where the liquid reaction medium can be recovered without great cost and complexity.

The finding of the present invention is that the substitution of polysaccharides or derivatives thereof can be carried out in a heterogeneous reaction regime using small amounts of ionic liquid.

The present invention is therefore directed to a process for the esterification, etherification or silylation of polysaccharide or derivatives thereof by means of an esterifying reagent, etherifying reagent or silylating reagent in the presence of an ionic liquid, in which the amount of ionic liquid is 2% to 24% by weight, based on the polysaccharide or derivatives thereof.

The process of the invention preferably refrains from a pretreatment step for activation, such as the use of an aqueous alkaline medium in the case of cellulose, for example. It is further desirable that the process is carried out heterogeneously.

Alternatively the present invention may also be described as follows:

Process for esterifying, etherifying or silylating polysaccharide or derivatives thereof by means of an esterifying reagent, etherifying reagent or silylating reagent in the presence of an ionic liquid, in which the process is carried out heterogeneously.

The process defined in the preceding paragraph is preferably carried out such that the amount of ionic liquid is 2% to 25% by weight, based on the polysaccharide or derivatives thereof. It is further preferred that there be no pretreatment step for activation in the process of the invention, such as the known use of an aqueous alkaline medium in the case of cellulose, for example.

As a result of the two process techniques indicated it is possible to carry out complete or virtually complete substitution of polysaccharides or derivatives thereof and, with a simple processing operation, to recover the ionic liquid and hence be able to use it again in a further process cycle. As a result of this new kind of use of ionic liquids in polysaccharide chemistry, the use of the toxic and environmentally hazardous ionic liquids is minimized, thereby also enabling derivatizations on a relatively large scale. Furthermore, the process of the invention makes it possible, particularly in the case of the synthesis of short-chain starch esters, to achieve high reagent yields, hence enabling access to precise target degrees of substitution. As a result, not only is the use of ionic liquid reduced, but there is also a reduction in the amount of substitution reagents such as etherifying reagents, esterifying reagents or silylating reagents, as compared with conventional synthesis procedures.

By heterogeneous operational regime or heterogeneous reaction conditions, the present invention means the reaction of polysaccharides and derivatives thereof which are not completely in solution in the ionic liquid. Before or during the esterification, etherification or silylation, the polysaccharides or derivatives thereof are in solution preferably to an extent of not more than 50% by weight, i.e. 10% to 50% by weight, more preferably not more than 40% by weight, i.e. 10% to 40% by weight, and even more preferably up to 30% by weight, i.e. 10% to 30%, based on the total weight of the batch.

A key feature of the present invention is that the ionic liquid, in contrast to known substitution processes in polysaccharide chemistry, is used in particularly small quantities. It is therefore preferred for the amount of ionic liquid to be not more than 30% by weight, preferably 2% to 30% by weight, with particular preference 2% to 25% by weight, even more preferably 2% to 20% by weight, with particular preference 2% to 15% by weight, such as 2% to 10% by weight, based on the polysaccharide or derivatives thereof.

Particularly good results can be achieved if the ionic liquid is added in a specific molar ratio relative to the anhydroglucose units (AGU) of the polysaccharide or derivatives thereof. One anhydroglucose unit indicates the amount of hydroxyl groups per glucose unit. For instance, one anhydroglucose unit of cellulose has three hydroxyl groups. Accordingly, it is preferred to use 0.016 to 1.35 mole equivalents, more preferably 0.017 to 1.30 mole equivalents, even more preferably 0.020 to 1.0 mole equivalents, with particular preference 0.08 to 0.90 mole equivalents, of ionic liquid per anhydroglucose unit of the polysaccharide or derivatives thereof.

The reaction temperature is preferably above the melting point of the ionic liquid, but preferably does not exceed 200° C. Particularly suitable temperatures are between 100 and 150°, more particularly between 120 and 135° C.

The reaction time is dependent in particular on the desired degree of substitution. The degree of substitution (DS) indicates the average number of hydroxyl groups reacted in an anhydroglucose unit. Accordingly, the higher the target degree of substitution, the longer the reaction time. In principle it is desirable for the reaction time to be not more than 24 hours, but preferably not more than 4 hours. Particularly suitable reaction times are between 30 minutes and 3.5 hours.

The amount of esterifying reagent, etherifying reagent or silylating reaction is likewise heavily dependent on the desired degree of substitution. On the other hand, a particular feature of the present invention is that, relative to the known substitution processes for polysaccharides, the amounts of substitution reagent to be used are fairly low. Accordingly, the amount of esterifying reagent, etherifying reagent or silylating reagent used is not more than 5.5 mole equivalents per anhydroglucose unit, more preferably not more than 4.5 mole equivalents per anhydroglucose unit, and in particular not more than 4.0 mole equivalents per anhydroglucose unit. In one particular embodiment, stoichiometric amounts of substituting reagents are used in relation to the anhydroglucose unit.

The present process is applicable in principle to all polysaccharides and derivatives thereof. It has emerged in particular that the present process of the invention is particularly suitable for the esterification, etherification or silylation of polysaccharides or derivatives thereof, selected from the group consisting of starch, cellulose, xylan and chitosan.

Ionic liquids are, in particular, salts which are liquid at temperatures below 100°. Preference is given to using ionic liquids selected from the group consisting of imidazolium compounds, pyridinium compounds, tetraalkylammonium compounds and mixtures thereof. Particularly preferred ionic liquids of the present invention are 1-N-butyl-3-methylimidazolium chloride, 1-N-allyl-3-methylimidazolium chloride and 1-N-ethyl-3-methylimidazolium chloride. The use of 1-N-butyl-3-methyl-imidazolium chloride has emerged as being especially advantageous.

With regard to the substitution reagents to be used, there are no particular limitations necessary. However, the use of esterifying reagents selected from the group consisting of C₁ to C₂₀ alkyl anhydrides, such as C₁ to C₆ alkyl anhydrides, and C₂ to C₂₁ alkanoyl chlorides, such as C₂ to C₆ alkanoyl chlorides, has proved to be particularly useful. Of these, acetic anhydride or propionic anhydride has been found to be especially appropriate.

Where an etherification is to be carried out, C₁ to C₂₀ alkyl epoxides in particular, such as C₁ to C₆ alkyl epoxides, have been found appropriate. Furthermore, the alkyl epoxides may also have additional functionalization. Thus, for example, it is preferred for the C₃ to C₈ alkyl epoxides to include at least one further functional group, selected from the group consisting of ether group, allyl group, vinyl group and quaternary nitrogen group. Of these, the allyl glycidyl ether has been found to be particularly suitable.

With regard to the silylation as well, all conceivable silylating reagents can be used. However, the use of a silylating reagent of the formula (I)

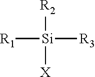

in which R₁, R₂ and R₃ independently of one another represent the radical selected from the group consisting of C₁ to C₁₂ alkyl, C₂ to C₁₂ alkenyl and C₂ to C₁₂ alkynyl, it being possible for these radicals likewise to comprise functional groups, and X is selected from the group consisting of —NH—SiR₄R₅R₆, —N(CH₂CH₃)₂ and —N═C(CH₃)—O—Si(CH₃)₃, where R₄, R₅ and R₆ independently of one another represent the radical selected from the group consisting of C₁ to C₁₂ alkyl, C₂ to C₁₂ alkenyl and C₂ to C₁₂ alkynyl,
appears to be particularly advantageous.

A silylating agent found particularly useful is 1,1,1,3,3,3-hexamethyldisilazane (HMDS).

The process of the invention can be carried, out under standard process conditions, i.e. in a reactor system. Alternatively, the substitution of the polysaccharides or derivatives thereof can be carried out in a microwave reactor.

The process of the invention is described in more precision below by the present examples, without being limited to said examples.

### EXAMPLES

### Ex. 1

### Synthesis of Starch Propionate—Use of 1.2 mol eq of BmimCl per AGU

Starch (dried at 105° C. for at least 15 hours) is heated together with 1.2 mol eq of 1-N-butyl-3-methylimidazolium chloride and 4.5 mol eq of propionic anhydride to 130° C. in a suitable round-bottom flask, by means of an oil bath, with stirring. When the reaction temperature is reached, the reaction time is 4 hours. When using 1.2 mol eq of BmimCl per AGU, the reaction mixture has a yellowish transparency after 3 hours. When the reaction time is at an end, the reaction solution is cooled to room temperature and the product is precipitated from ethanol. It is washed with ethanol a number of times and dried under reduced pressure.

The product is starch-propionate having a degree of substitution of 2.9 (determined by ¹³C-NMR), and is soluble in acetone, ethyl acetate and dichloromethane but insoluble in water and ethanol.

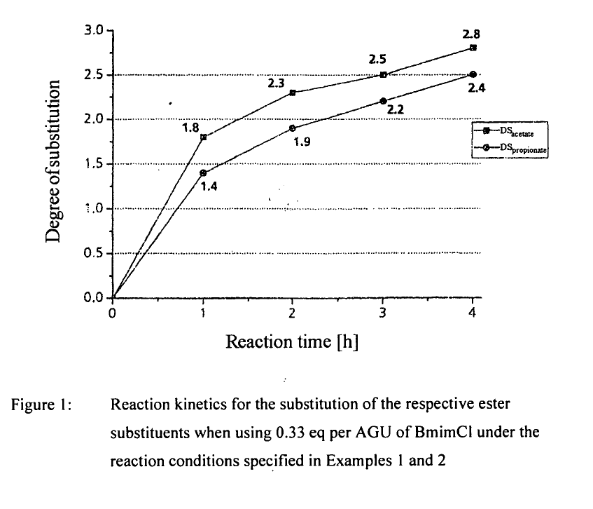

In analogy to the synthesis elucidated in Example 1, further syntheses were carried out for the preparation of starch propionates, with the concentration of BmimCl being varied. The results are set out in Table 1, and the reaction kinetics for the use of 0.33 mol eq of BmimCl per AGU as catalyst are set out in FIG. 1.

TABLE 1
Degrees of substitution achieved for starch propionate in response to
changes in the concentration of BmimCl under synthesis conditions as
described in Ex. 1 (with 4.5 mol eq of anhydride). (Process as Ex. 1;
only fraction of BmimCl changed)

| mol eq of BmimCl | |
|---|---|
| per AGU | Σ DSₚᵣₒₚᵢₒₙₐₜₑ** |
| 0.5 | 2.8 |
| 0.33 | 2.2 |
| 0.15 | 1.5 |
| 0.075 | 0.7 |

**Determined by titration in accordance with D. Klemm, B. Philipp, T. Heinze, U. Heinze, W. Wagenknecht: Comprehensive Cellulose Chemistry, Wiley-VCH, Volume 1, 1998, Appendix p. 235

### Ex. 2

### Synthesis of Starch Acetate—Use of 0.33 mol eq of BmimCl per AGU

Starch (dried at 105° C. for at least 15 hours) is heated together with 0.33 mol eq of 1-N-butyl-3-methylimidazolium chloride and 4.5 mol eq of acetic anhydride to 130° C. in a suitable reactor with stirring. When the reaction temperature is reached, the reaction time is 4 hours. After the reaction time is at an end, the reaction solution is cooled to room temperature and the product is precipitated from ethanol. It is washed a number of times with ethanol and dried under reduced pressure. The product is starch acetate having a degree of substitution of 2.8, which is soluble in acetone, ethyl acetate and dichloromethane but insoluble in water and ethanol. The kinetics of the esterification are shown in FIG. 1.

Table 2 shows the degrees of substitution achieved in the acetate substituents when the molar equivalents of BmimCl per anhydroglucose unit are varied.

TABLE 2
Degrees of substitution achieved for starch acetates when the
concentration of BmimCl is changed under synthesis conditions as
described in Ex. 2 (with 4.5 mol eq of anhydride).

| mol eq of BmimCl per AGU | Σ DS_{acetate}** |
|---|---|
| 0.33 | 2.8 |
| 0.15 | 1.8 |
| 0.075 | 1.5 |

### Ex. 3

### Synthesis of Starch Propionate—Use of Air-Dry Starch

In analogy to the synthesis procedure described in Ex. 1, air-dry starch (solids content 88.9%) is reacted with 4.5 mol eq of propionic anhydride per AGU and with 0.33 mol eq of 1-N-butyl-3-methylimidazolium chloride per AGU. The product is starch propionate having a degree of substitution of 2.0, which is soluble in acetone, ethyl acetate and dichloromethane but insoluble in water and ethanol.

### Ex. 4

### Synthesis of Starch Acetate—Use of 0.075 eq of BmimCl per AGU: 24 h Reaction Time

0.075 mol eq of 1-N-butyl-3-methylimidazolium chloride and 4.5 mol eq of acetic anhydride are heated together with starch (dried at 105° C. for at least 15 hours) to 130° C. in a suitable round-bottom flask with stirring. When the reaction temperature is reached, the reaction time is 24 hours. When the reaction time is at an end, the brown, homogeneous reaction solution is cooled to room temperature and the product is precipitated from ethanol. It is washed a number of times with ethanol and dried under reduced pressure.

The product is a starch acetate having a degree of substitution of 3.0, which is soluble in acetone, ethyl acetate and dichloromethane but insoluble in water and ethanol.

In accordance with the reaction regime described in Example 4, the molar equivalents of BmimCl were reduced further and the degree of substitution was investigated as a function of the amount of BmimCl. The results are summarized in Table 3.

TABLE 3
Degrees of substitution achieved after increase in reaction time

| mol eq of BmimCl per AGU | Σ DS_{acetate}** |
|---|---|
| 0.075 | 3.0 |
| 0.0375 | 2.6 |
| 0.01875 | 0.6 |

### Ex. 5

### Synthesis of Starch Acetate—Change in Reaction Temperature

The synthesis is carried out in the same way as in the instructions for Ex. 2, but with the reaction temperature lowered to 85° C. The product is a starch acetate having a degree of substitution of 0.6.

### Ex. 6

### Synthesis of Starch Acetate by Esterification with Acetic Acid

Starch (dried at 105° C. for at least 15 hours) is heated together with 0.15 mol eq of 1-N-butyl-3-methylimidazolium chloride and 6 mol eq of acetic acid to 130° C. in a suitable reactor with stirring. When the reaction temperature is reached, the reaction time is 18 hours. When the reaction time is at an end, the reaction solution is cooled to room temperature and the product is precipitated from ethanol. It is washed a number of times with ethanol and dried under reduced pressure. The starch acetate obtained possesses a degree of substitution of 0.5.

### Ex. 7

### Synthesis of Starch Acetate—Variation in the Mole Equivalents of Ac₂O

For this purpose, the synthesis is carried out as described in Ex. 2, and first of all only the molar equivalents of acetic anhydride per anhydroglucose unit are varied. Further experiments with a reduced amount of anhydride and IL followed these experiments. The amounts used and degrees of substitution achieved are summarized in Table 4.

TABLE 4
Degrees of substitution achieved after reducing the amount of
Ac₂O used

| mol eq | mol eq of | | |
|---|---|---|---|
| of Ac₂O | BmimCl | | Homogeneous |
| per AGU | per AGU | ΣDS_{acetate}** | reaction batch |
| 4.5 | 0.33 | 2.8 | − |
| 3.25 | 0.33 | 2.8 | + |
| 2.5 | 0.33 | 2.6 | + |
| 2.5 | 0.19 | 2.5 | + |
| 1.5 | 0.19 | 1.7 | + |

### Ex. 8

### Synthesis of Starch Acetate—Variation in the Ionic Liquid

For this purpose, starch (dried at 105° C. for at least 15 hours) together with 0.33 mol eq per AGU of the respective ionic liquid is reacted with 4.5 mol eq per AGU of acetic anhydride, as described in Ex. 2.

The results are contained in Table 5.

TABLE 5
Degrees of substitution achieved for
starch acetates using different ionic liquids

| Ionic liquid | ΣDS_{acetate}** |
|---|---|
| BmimCl | 2.8 |
| TBACl | 0.8 |

### Ex. 9

### Synthesis of Starch Acetates in a Microwave Reactor

In a microwave reactor, starch (dried at 105° C. for at least 15 hours) is reacted with acetic anhydride and BmimCl to give starch acetate. Heating in this case takes place to an internal temperature of 130° C. over the course of 10 minutes, and cooling takes place to room temperature over the course of 30 minutes. More precise reaction conditions and results are given in Table 6.

TABLE 6
Degrees of substitution achieved using a microwave heating/
cooling unit

| mol eq of | mol eq of | | |
|---|---|---|---|
| Ac₂O | BmimCl | | |
| per AGU | per AGU | t_{reaction}[h] | ΣDS_{acetate}** |
| 4.0 | 0.056 | 4 | 1.1 |
| 3.25 | 0.0375 | 2 | 0.4 |

### Ex. 10

### Synthesis of allyl-2-hydroxypropylstarch

0.1 mol eq of BmimCl per AGU and 7 mol eq of allylglycidyl ether per AGU are heated together with starch (dried at 105° C. for at least 15 hours) to 100° C. in a suitable round-bottom flask with stirring. When the reaction temperature is reached, the reaction time is 4 hours. When the reaction time is at an end, the reaction batch is cooled to room temperature and the product is precipitated from ethanol. It is washed a number of times with ethanol and dried under reduced pressure. The product is allyl-2-hydroxypropylstarch having a degree of substitution of 0.2.

### Ex. 11

### Synthesis of Cellulose Acetates

For the esterification reaction of cellulose to cellulose acetates, a wide variety of reaction conditions are tested. One reaction regime entails the reaction of microcrystalline cellulose (DP_{Cuen}=260; dried at 105° C., >15 h) with 1.2 mol eq of BmimCl and 8.7 mol eq of acetic anhydride per AGU at a temperature of 130° C. in a suitable reactor for 2 hours. Subsequently, the product in the reaction batch, which has been cooled to room temperature, is precipitated from ethanol and washed to neutrality. The degree of substitution of acetate groups found is 1.2. Table 7 shows further reaction conditions and degrees of substitution achieved for various batches in the preparation of cellulose acetate.

TABLE 7
Reaction conditions and degrees of substitution achieved for the
preparation of cellulose acetates.

| mol eq of | | | | |
|---|---|---|---|---|
| BmimCl/ | mol eq of | | | |
| AGU | Ac₂O/AGU | T [° C.] | t [h] | ΣDS_{acetate}** |
| 0.8 | 5.8 | 130 | 6 | 1.6 |
| 0.15 | 4.5 | 130 | 24 | 1.2 |
| 0.15 | 9.0 | 130 | 24 | 1.1 |

### Ex. 12

### Synthesis of Methylcellulose Acetate

Methylcellulose (Methocel®, Methoxy content 27.5%-32%) is introduced together with 10.6 mol eq of acetic anhydride into a suitable reactor and admixed with 0.1 mol eq of BmimCl per anhydroglucose unit. The reaction batch is heated to 130° C. and then the reaction is continued at this temperature for 4 hours. After cooling to room temperature, the product is precipitated from an aqueous medium and washed to neutrality. The product is methylcellulose acetate having a DS_{acetate}=0.6, which is insoluble in water or acetone but soluble in DMSO.

### Ex. 13

### Synthesis of Trimethylsilylcellulose

Trimethylsilylcellulose is obtained by the reaction of microcrystalline cellulose (DP_{Cuen}=260; dried at 105° C., >15 h) with 1,1,1,3,3,3-hexamethyldisilazane (HMDS) using BmimCl as catalyst at 125° C. in a round-bottom flask. The reaction batch was subsequently precipitated from ethanol and worked up. More precise experimental conditions and results are set out in Table 8.

TABLE 8
Reaction conditions and degrees of substitution achieved for the
preparation of trimethylsilylcelluloses

| mol eq of | mol eq of | | |
|---|---|---|---|
| BmimCl/AGU | HMDS/AGU | t[h] | ΣDS_{TMS}*** |
| 0.33 | 4.6 | 18 | 0.8 |
| 0.1 | 5.6 | 18 | 0.8 |
| 0.28 | 5.6 | 6.5 | 0.9 |

***Determined by means of solid-state ¹³C-NMR

All trimethylsilylcelluloses shown are soluble in dichloromethane.

### Ex. 14

### Synthesis of Chitosan Propionate

Chitosan (degree of deacetylation: 90%; dried at 105° C. for 15 h) and 7.5 mol eq of propionic anhydride per AGU are placed together with 0.5 mol eq of 1-N-butyl-3-methylimidazolium chloride per AGU in a suitable reactor and heated to 130° C. Subsequently, 15 minutes after the reaction temperature has been reached, a further 3.3 mol eq of propionic anhydride per AGO are added to the reaction mixture. The reaction time is 24 hours. When the reaction time is at an end, the brown reaction mixture is cooled to room temperature and introduced into ethanol. The product is washed a number of times with ethanol and dried under reduced pressure. This gives a yellowish product having a DSₚᵣₒₚᵢₒₙₐₜₑ=1.5***.

### Ex. 15

### Synthesis of Xylan Esters

Xylan (dried at 105° C. for 15 h) is reacted with 0.33 mol eq of BmimCl per AGU with the corresponding anhydride at 130° C. for 4 hours. Table 9 shows the degrees of substitution and reagent yields achieved.

TABLE 9
Synthesis conditions and results for xylan esterification

| | mol eq of | | |
|---|---|---|---|
| | anhydride | | Reagent yield |
| Anhydride | per AGU | ΣDₑₛₜₑᵣ*** | [%] |
| Acetic anhydride | 7.0 | 1.7 | 25 |
| Propionic | 6.0 | 1.1 | 18 |
| anhydride | | | |

1. Process for esterifying, etherifying or silylating polysaccharide or derivatives thereof by means of an esterifying reagent, etherifying reagent or silylating reagent in the presence of an ionic liquid, in which
(a) the process is carried out under heterogeneous reaction conditions, and
(b) the amount of ionic liquid is 2% to 25% by weight, based on the polysaccharide or derivatives thereof.

2. Process according to claim 1, in which 0.017 to 1.3 mole equivalents of ionic liquid are used per anhydroglucose unit (AGU) of the polysaccharide or derivatives thereof.

3. Process according to claim 1, in which the polysaccharide or derivatives thereof is or are completely in solution neither before nor during the esterification, etherification or silylation.

4. Process according to claim 1, in which the polysaccharide or derivatives thereof is or are in solution to an extent of not more than up to 50% by weight, based on the total weight of the batch, before or during the esterification, etherification or silylation.

5. Process according to claim 1, in which the reaction temperature is above the melting point of the ionic liquid but not more than 200° C.

6. Process according to claim 1, in which the reaction time is not more than 24 hours.

7. Process according to claim 1, in which the polysaccharide or derivatives thereof is or are selected from the group consisting of starch, cellulose, xylan and chitosan.

8. Process according to claim 1, in which the ionic liquid is selected from the group consisting of imidazolium compounds, pyridinium compounds, tetraalkylammonium compounds and mixtures thereof.

9. Process according to claim 1, in which the esterifying reagent is selected from the group consisting of C₁ to C₂₀ alkyl anhydrides and C₂ to C₂₁ alkanoyl chlorides.

10. Process according to claim 1, in which the etherifying reagent is a C₃ to C₈ alkyl epoxide.

11. Process according to claim 1, in which the etherifying reagent is a C₃ to C₈ alkyl epoxide and this alkyl epoxide besides the epoxides comprises at least one further functional group selected from the group consisting of ether group, allyl group, vinyl group and quaternary nitrogen group.

12. Process according to claim 1, in which the silylating reagent has the formula (I) 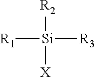
in which
R₁, R₂ and R₃ independently of one another represent the radical selected from the group consisting of C₁ to C₁₂ alkyl, C₂ to C₁₂ alkenyl and C₂ to C₁₂ alkynyl, and
X is selected from the group consisting of —NH—SiR₄R₅R₆, —N(CH₂CH₃)₂ and —N═C(CH₃)—O—Si(CH₃)₃, where R₄, R₅ and R₆ independently of one another represent the radical selected from the group consisting of C₁ to C₁₂ alkyl, C₂ to C₁₂ alkenyl and C₂ to C₁₂ alkynyl.

13. Process according to claim 1, in which the process is carried out in a microwave reactor.
